(19) United States
(12) Patent Application Publication
(10) Pub. No.: US 2007/0287123 A1
(43) Pub. Date: Dec. 13, 2007

(54) Mouth mirror handle featuring improved lens mounting technique

(76) Inventors: Ronald L. Swift, Medina, MN (US); James M. Campion, Buffalo, MN (US)

Correspondence Address: David George Johnson, Post Office Box 286, Aitkin, MN, 56431, US

(21) Appl. No.: 11/451,877

(22) Filed: Jun. 12, 2006

(51) Int. Cl. A61B 1/24; B29D 11/00

(52) U.S. Cl. 433/30; 264/1.9

(57) ABSTRACT

A method of manufacturing a mirror assembly (14) by placing a beveled mirror (18) within a carrier (19) prior to inserting the carrier into a mirror handle head (23). The mirror handle head (23) is inserted into a mold position (27) and a molten thermoplastic resin is introduced into the mold. A series of mirror retaining clips (20) formed integrally with the carrier (19) maintain the mirror (18) in a precise location during the molding process and permit resin to overlay the mirror bevel (35) without reaching the mirror surface (22). The mirror handle (15) may be customized during manufacture to include a ribbed, soft grip region (16, 17) by forming a handle core (36) that includes a recessed region defined by shoulders (43, 44). A central region of the core (36) includes voids that define characters or symbols (38, 39, 40, and 41) that are filled with a thermoplastic material during a molding process (4). Alternatively, the handle (15) may be formed during a single molding process and attached to the handle head (23) during formation of the mirror retention lip (49).

### FIELD OF THE INVENTION

The present invention relates generally to the field of dental examination mirrors, and more specifically to the field of manufacturing a mirror handle adapted to retain a mirror housed in a carrier.

### BACKGROUND

Hand held mirrors are commonly used by dentists and other medical practitioners to permit viewing of otherwise obscured dental surfaces and gums within the mouth of a patient. Dental mirrors are usually formed as circular mirrors that are attached to one end of a handle. The mirror handles are sometimes formed of metal, often with grooves or knurling to improve the ability of a dentist to securely grip the handle. An example of such a mirror is disclosed in U.S. Pat. No. 4,090,506, entitled DIAGNOSTIC INSTRUMENT, which was issued on May 23, 1978 to Pilgrim. Metal handles are durable, but are often uncomfortable within the mouth of a patient due to the cold feel of the metal and abrasiveness caused by handle surface tool work. Modem dental mirrors are more frequently formed of plastic, which is lighter and more easily manufactured using some type of molding process.

Plastic mirror handles can suffer from several drawbacks. The handle molding process can introduce longitudinal mold parting lines that sometimes create sharp ridges which are sensed by the dental patient. The choice of material can also be problematic, since some plastics such as general purpose styrene and ABS do not autoclave well. Polyester does not have sufficient strength to serve as a mirror handle, while a Nylon/Glass mixture is too brittle. Delrin does not meet the aesthetic standards of the dental industry, while Teflon is relatively expensive and difficult to mold.

An example of a mouth mirror is disclosed in U.S. Pat. No. 6,544,036, entitled “ILLUMINATING DENTAL/MEDICAL EXAMINATION MIRROR”, issued to Brattesani on Apr. 8, 2003. The Brattesani device includes a neck and head made of Lexan. A mirror is mounted within the head and retained by a lip or an adhesive. The actual method of manufacturing such a mirror is not disclosed, especially in a mass production environment. The disclosed lip retention method appears to require a separate staking process in order to place the mirror into the head.

In view of the foregoing, a need exists for a mouth mirror that can be manufactured in quantity and at a commercially viable cost. The mouth mirror must be easily gripped and manipulated by a dentist and be comfortable within the mouth of a patient.

### SUMMARY OF THE INVENTION

The present invention is a mouth mirror having an improved handle as well as an improved method for manufacturing the handle of a dental mirror. The present invention includes a mirror lens which is placed into a carrier that is separate from the handle. The carrier is then placed into the handle mold. Plastic is injected into the carrier/lens combination. The configuration of the carrier prevents the injected plastic from covering or contacting the reflective surface of the lens. The carrier also prevents the lens from moving during the injection process, thereby avoiding lens marring. The mirror includes a beveled sidewall that is covered and sealed by the injected plastic.

The handle is also formed from two separate components that are formed and joined using multiple plastic injection steps. The handle includes a flexible, widened portion formed to include a series of radial grooves. The flexible portion is formed and joined to the existing handle body during an injection molding process. The present method reduces parting lines or molding gate vestiges while improving the look and feel of the resulting mouth mirror.

### BRIEF DESCRIPTION OF THE DRAWINGS

FIG. 1 is a left side elevation of a first embodiment of a mirror handle illustrating the principles of the present invention;

FIG. 2 is a perspective view of a complete mirror assembly utilizing a second embodiment of a mirror handle constructed according to the principles of the present invention;

FIG. 3 is a bottom plan view of the handle illustrated in FIG. 1;

FIG. 4 is a rear elevation of the mirror handle depicted in FIG. 1;

FIG. 5 is a perspective view illustrating the fabrication of a complete mirror assembly; and

FIG. 6 is an exploded perspective view of the mirror assembly illustrated in FIG. 2.

### DETAILED DESCRIPTION OF THE INVENTION

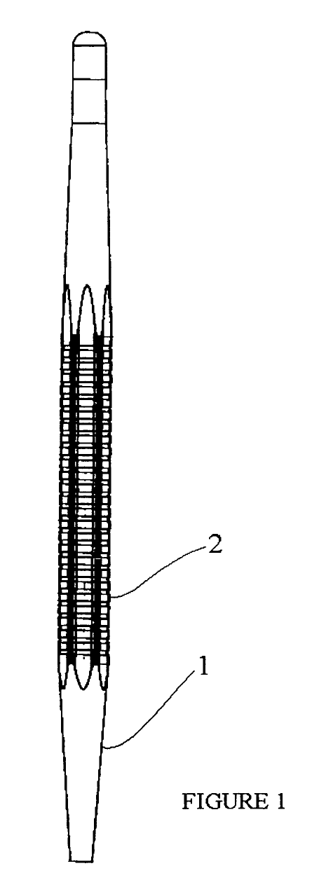

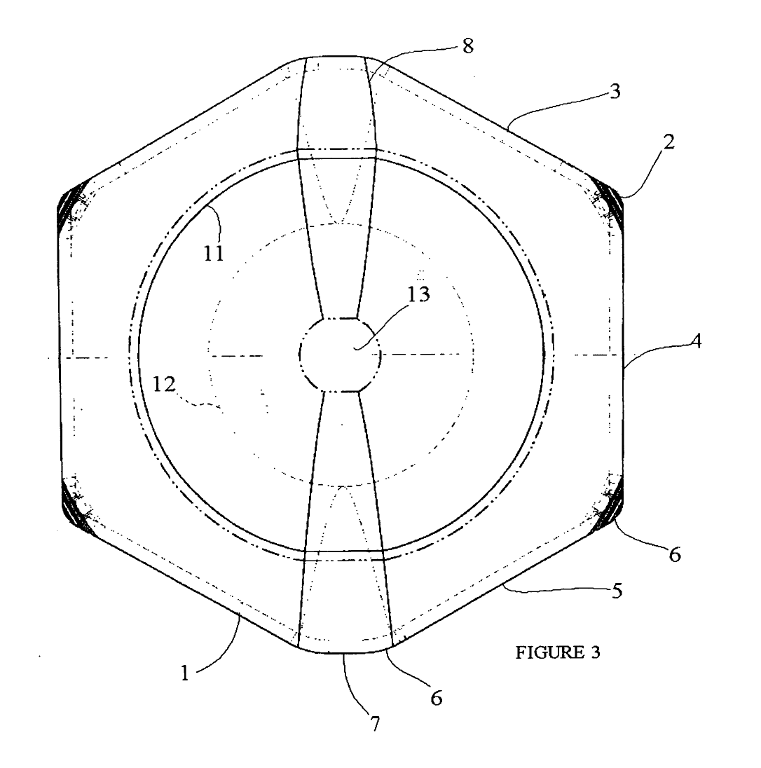

The present invention is designed and intended for use by a dentist or other health professional to facilitate an examination of the mouth, teeth and gums of a patient. Referring to FIGS. 1 and 3, a handle 1 is depicted which may be gripped and manipulated by the user. The handle 1 includes a ribbed or knurled region 2 which assists the user, typically a dentist, in maintaining a grip on the handle during the examination or treatment of a patient. The handle I is formed of a suitable thermoplastic material such as polycarbonate or preferably a general purpose polypropylene containing approximately twenty percent glass. The basic criteria for the thermoplastic material are that it must be autoclavable and maintain the shape of handle 1 without being excessively brittle or flexible. The handle 1 must not be easily broken and of course cannot be so expensive as to be prohibitive to the end user.

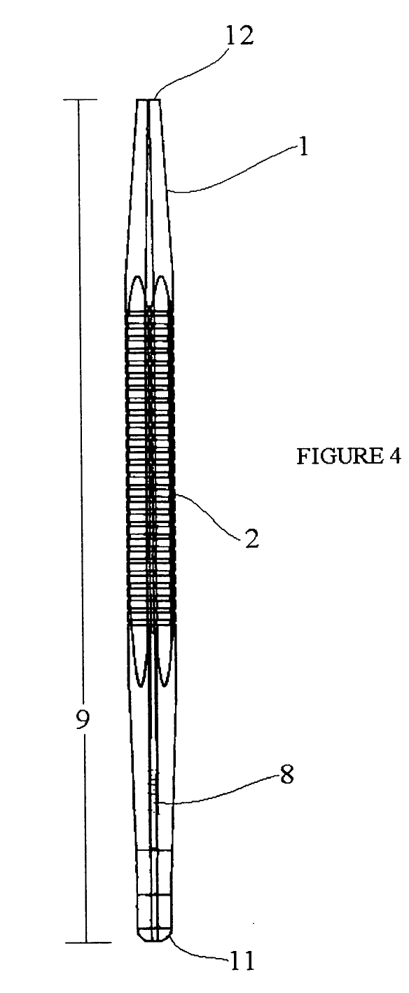

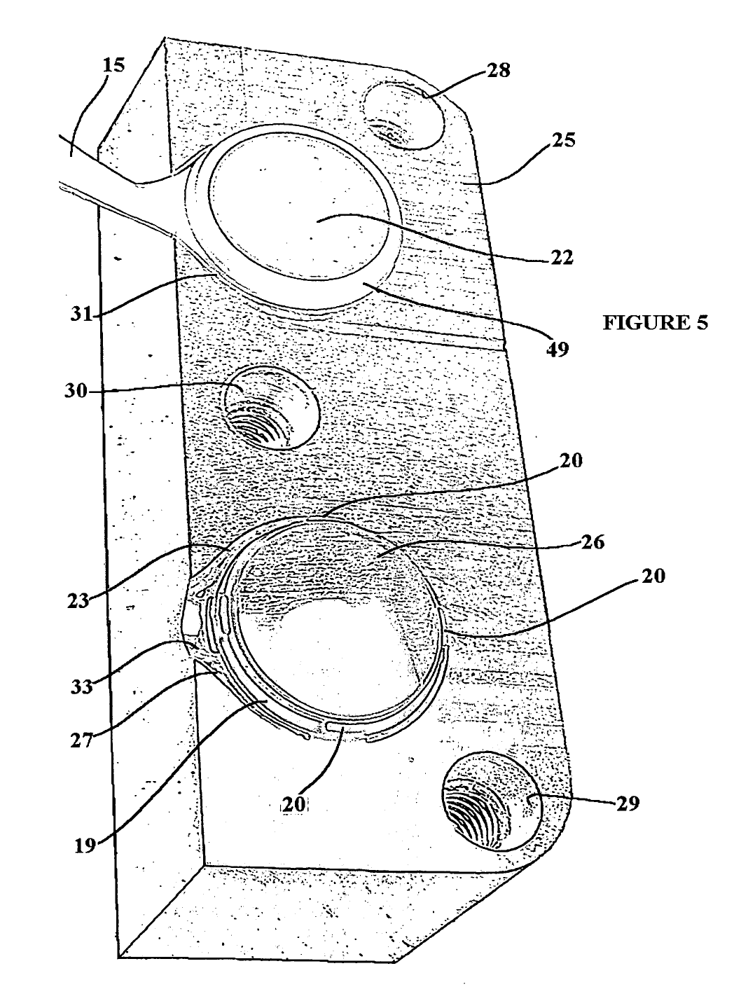

In one preferred embodiment of the present invention, the handle has a substantially hexagonal cross section with substantially flat sides 3, 4 and 5, for example. Each side 3, 4, 5 is joined to an adjacent side via a radius 6 and a relative short flattened section 7. Referring also to FIGS. 4 and 5, a longitudinal groove 8 extends for substantially the entire length 9 of the handle 1. The handle 1 includes a rounded base 11 terminating at button 13, and an opposite, substantially flat forward tip region 12.

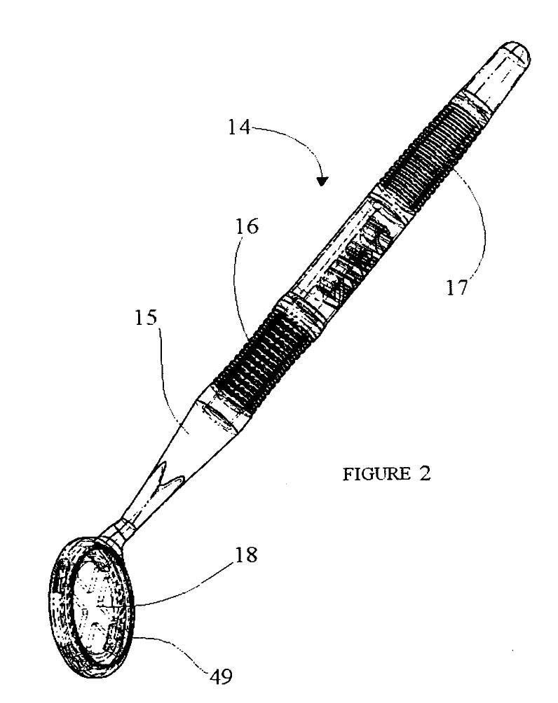

Referring also to FIG. 2, a complete mirror assembly 14 utilizing a second embodiment of the handle 15 is depicted. The handle 15 includes a tapered, ribbed portion 16 intended for contacting the index finger of the user. A rear ribbed portion 17 is formed on the handle surface to contact the hand of the user in an area between the fingers and the wrist. The ribbed portions 16 and 17 are preferably formed of a flexible thermoplastic material having a durometer of approximately thirty five. The mirror assembly 14 includes a mirror 18 having a multilayered Rhodium coating.

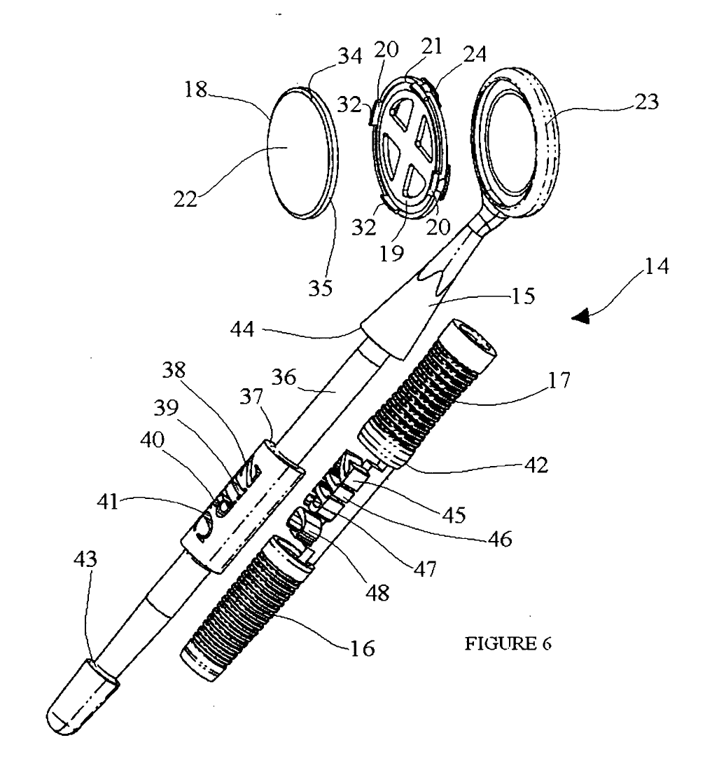

Referring also to FIG. 6, the mirror 18 is seen to be substantially circular in shape and has a thickness 22. The mirror 18 is formed to include a perpendicular sidewall 34 and a beveled sidewall 35. The beveled sidewall 35 interconnects the perpendicular sidewall 34 and the reflecting surface 22 of the mirror 18. The mirror 18 resides within a thermoplastic carrier 19. A series of upper mirror clips 20 are formed on the periphery 21 of the carrier 19. The mirror clips 20 retain the mirror 18 and have a distal portion including an upper surface 32 that extends just below the plane defined by the reflecting surface 22 of the mirror 18. The carrier 19 also includes a series of handle head clips 24 which are formed on the periphery 21. The handle head clips 24 are formed to engage a handle head 23 which is affixed to the handle 15.

Referring also to FIG. 5, the method of fabricating the mirror assembly 14 can be understood. Initially, the mirror carrier 19 is premolded to assume a shape adapted to retain the mirror 18. The mirror 18 is then placed within the carrier 19 to create a subassembly. The surface 22 of mirror 18 may optionally be covered with a protective tape or cover 26 that is later removed when the mirror assembly 14 is completed. The combination of mirror 18 and carrier 19 is placed within the handle head 23. The handle head 23 which contains both the carrier 19 as well as the mirror 18 is placed within the mold half 25. At this point in the assembly process the handle head 23 has typically not yet been affixed to the mirror handle 2 or 15, as is the case for the carrier 19 residing in mold position 27. The end 12 of the mirror handle 2 or 15 is placed within the depression 33 in each mold position 27 and 31. A second, mating mold half (not shown) is placed over the mold half 25 to create a complete moldset which is held in place by threaded fasteners placed in threaded bores 28, 30 and 29. Once the mold halves are secured, a molten thermoplastic resin is injected into each mold position 27 and 31. The moldset is formed so as to allow the molten thermoplastic resin to reach the distal or upper portion 32 of each retaining clip, that is, the resin rises to a level that covers the flat upper surface 32 of each clip 20. After covering the level of surface 32, the beveled surface 35 of the mirror 18 is covered by the resin while the mirror surface 22 remains slightly above (on the order of 0.001 inch) the level of the resin. The carrier 19 holds the mirror 18 in place during the molding process and the cured resin ultimately holds the mirror 18 in place by forming a lip 49 which overlies the beveled surface 35. The carrier 19 and the handle 15 are fully bonded to the head 23 once the resin cures.

As seen in FIG. 6, the handle assembly 14 can also be formed by a similar over molding process. The handle 15 includes a core 36 formed of a first plastic material during a premolding process. In the example illustrated, the core includes a widened cylindrical region 37 into which voids 38,39, 40 and 41 are formed which can signify a company logo or brand name. The handle body 42 is formed by surrounding the core 36 with a mold (not shown) that extends between shoulders 43 and 44 and which substantially surrounds and isolates the core region 36. A second thermoplastic material is injected into the mold, forming the ribbed regions 16 and 17 as well as filling the voids 37 through 41, thereby creating the letters or symbols 45, 46, 47 and 48 that are defined by the voids.

Although the present invention has been described with reference to the foregoing examples, changes can be made to these specific embodiments without departing from the principles of the invention. For example, while the mirror assembly 14 is shown as a composite of two discrete molding processes, more molding steps can be introduced to create a more complex mirror assembly. Similarly, different materials may be used in forming the mirror assembly 14 depending on cost, strength, durability and aesthetic requirements. Thus, many other mirror configurations are possible, and the scope of the present invention may be appreciated only by reference to the claims.

1. A method of manufacturing an article including a premanufactured optical surface, comprising the steps of:
forming a beveled sidewall on the premanufactured optical surface;
forming a carrier adapted to retain the premanufactured optical surface in a predetermined position;
inserting the premanufactured optical surface into the carrier;
placing the carrier containing the premanufactured optical surface into a first moldset;
injecting a molten thermoplastic resin into the first moldset so as to bond the premanufactured optical surface to the carrier.

2. The method of claim 1, further comprising the step of preventing the molten thermoplastic resin from contacting a designated region of the premanufactured optical surface.

3. The method of claim 2, further comprising the steps of:
forming a plurality of retaining clips on the carrier such that a distal portion of each retaining clip resides in a plane defined by the designated region of the premanufactured optical surface; and
forming the moldset so as to prevent the molten thermoplastic resin from reaching the distal portion of each retaining clip.

4. The method of claim 3, further comprising the step of forming a lip of thermoplastic material adjacent to a peripheral region of the premanufactured optical surface.

5. The method of claim 4, further comprising the steps of:
placing the carrier into a handle head; and
affixing the handle head to a handle so as to permit manipulation of the premanufactured optical surface by a user.

6. The method of claim 5, further comprising the step of forming the handle so as to include a core region.

7. The method of claim 6, further comprising the steps of:
placing the core region into a second moldset that substantially surrounds and isolates the core region; and
injecting a thermoplastic material into the second moldset so as to form a grip around the core region, thereby facilitating manipulation of the premanufactured optical surface by a user.

8. The method of claim 7, further comprising the steps of:
forming the core region of a first type of thermoplastic material; and
forming the grip of a second type of thermoplastic material.

9. The method of claim 8, further comprising the steps of:
forming the core so as to include a relatively enlarged central region;
forming a plurality of voids within the central region; and
filling the plurality of voids with the second type of thermoplastic material while forming the grip.

10. The method of claim 9, further comprising the step of forming the plurality of voids with in the central region so as to create a graphical design surrounded by the enlarged central region.

11. (canceled)

12. (canceled)

13. (canceled)

14. (canceled)

15. (canceled)

16. (canceled)

17. A method of manufacturing an examination mirror comprising the steps of:
placing a mirror in a mirror retaining carrier;
inserting the carrier into a mirror handle head;
placing the carrier into a moldset; and
injecting a thermoplastic resin into the moldset so as to bond the carrier to the mirror and the handle head.

18. The method of claim 17, further comprising the step of forming a beveled sidewall adjacent to a reflecting surface of the mirror.

19. The method of claim 18, further comprising the step of forming the moldset so as to create a thermoplastic lip abutting the beveled sidewall of the mirror and thereby retaining the mirror between the lip and the carrier.

20. The method of claim 19, further comprising the step of placing a protective cover over the reflecting surface of the mirror so as to prevent damage to the mirror within the moldset.
